(19) United States
(12) Patent Application Publication
(10) Pub. No.: US 2019/0322965 A1
(43) Pub. Date: Oct. 24, 2019

(54) PROCESS FOR FLUSHING AN OLIGOMERIZATION REACTOR AND OLIGOMERIZATION OF AN OLEFIN

(71) Applicant: SABIC GLOBAL TECHNOLOGIES B.V., Bergen op Zoom (NL)

(72) Inventors: Abdullah Alqahtani, Riyadh (SA); Shahid Azam, Riyadh (SA); Shehzada Khurram, Riyadh (SA); Anina Wöhl, Munich (DE); Wolfgang Müller, Munich (DE); Andreas Meiswinkel, Munich (DE); Heinz Bölt, Munich (DE); Ralf Noack, Munich (DE); Marco Harff, Munich (DE); Gabriel Waurick, Munich (DE); Tobias Meier, Munich (DE)

(21) Appl. No.: 15/780,756

(22) PCT Filed: Dec. 1, 2016
(86) PCT No.: PCT/IB2016/057273

§ 371 Date: Jun. 1, 2018

Related U.S. Application Data
(60) Provisional application No. 62/263,156, filed on Dec. 4, 2015.

(51) Int. Cl. C11D 7/50; C11D 7/24; C11D 11/00; C07C 2/08

(52) U.S. Cl. CPC C11D 7/5027; C07C 2/08; C11D 11/0041; C11D 7/241

(57) ABSTRACT

A process for flushing an oligomerization reactor is described, including flushing the oligomerization reactor with a flushing medium comprising C₆ linear alpha olefins, C₈ linear alpha olefins, C₁₀ linear alpha olefins, or a combination including at least one of the foregoing, to provide a purge stream comprising the C₆ linear alpha olefins, C₈ linear alpha olefins, C₁₀ linear alpha olefins, or a combination including at least one of the foregoing, and a polymer byproduct of an oligomerization reaction. A process for the oligomerization of an olefin is also described, wherein the process includes oligomerizing the olefin in the reactor to form a reaction product stream comprising linear alpha olefins, and subsequently flushing the oligomerization reactor according to the process disclosed herein.

### BACKGROUND

Methods for oligomerization of ethylene and other alpha olefins are widely known. Such oligomerization methods include oligomerizing the monomer (e.g., ethylene) in a reactor in the presence of catalyst, co-catalyst and solvent. Following oligomerization in the reaction, product material comprising oligomer and/or polymer, non-reacted monomer(s), catalyst, co-catalyst and solvent can be discharged from the reactor and can be further processed. Suitable further processing can include catalyst deactivation, separation of solvent and oligomer product, and oligomer fractionation. The oligomerization methods can be operated continuously such that unreacted monomer or solvent can be recirculated in the oligomerization plant.

From time to time, the oligomerization reactors and other plant equipment require cleaning, as fouling or plugging can occur at the reactor walls and the walls of the piping. Cleaning can be achieved by flushing the piping and the reactor equipment with a flushing medium, preferably at an elevated temperature. The flushing medium is generally any suitable solvent. Flushing the piping and reactor equipment produces significant quantities of contaminated flushing medium to be disposed of. Disposing of the contaminated flushing medium is often difficult and cost-intensive.

Accordingly, there is a continuing need for a process for flushing an oligomerization reactor that can overcome the above-described limitations. Specifically, a suitable process will avoid a costly imported flushing medium, and the use of a flushing medium that requires the installation of additional reprocessing equipment and disposal through difficult and cost-intensive means, and further can adequately remove fouled reaction components from the reactor equipment.

### BRIEF DESCRIPTION

Disclosed in various embodiments are processes for flushing an oligomerization reactor and processes for the oligomerization of an olefin.

A process for flushing an oligomerization reactor comprises: flushing the oligomerization reactor with a flushing medium comprising C₆ linear alpha olefins, C₈ linear alpha olefins, C₁₀ linear alpha olefins, or a combination comprising at least one of the foregoing, to provide a purge stream comprising the C₆ linear alpha olefins, C₈ linear alpha olefins, C₁₀ linear alpha olefins, or a combination comprising at least one of the foregoing, and a polymer byproduct of an oligomerization reaction.

A process for flushing an oligomerization reactor comprises: flushing the oligomerization reactor with a flushing medium comprising C₆ linear alpha olefins, C₈ linear alpha olefins, C₁₀ linear alpha olefins, or a combination comprising at least one of the foregoing, to provide a purge stream comprising the C₆ linear alpha olefins, C₈ linear alpha olefins, C₁₀ linear alpha olefins, or a combination comprising at least one of the foregoing, and a polymer byproduct; contacting, in a separation system, the purge stream with a C₈+ product fraction from an olefin oligomerization process to form a first intermediate stream; separating the first intermediate stream to provide a C₆₋₁₀ linear alpha olefin fraction, and a heavy stream comprising a C₁₂+ product fraction and the polymer byproduct; and recycling at least a portion of the C₆₋₁₀ linear alpha olefin fraction to a reactor flushing system.

A process for oligomerization of an olefin comprises: oligomerizing the olefin in the reactor to form a reaction product stream comprising linear alpha olefins; separating a C₄₋₆ linear alpha olefin product fraction from the reaction product stream to provide a first stream comprising a C₈+ product fraction; subsequent to oligomerizing the olefin in the reactor, flushing the reactor with a flushing medium comprising C₆ linear alpha olefins, C₈ linear alpha olefins, C₁₀ linear alpha olefins, or a combination comprising at least one of the foregoing, to provide a purge stream comprising the C₆ linear alpha olefins, C₈ linear alpha olefins, C₁₀ linear alpha olefins, or a combination comprising at least one of the foregoing, and a polymer byproduct; contacting in a separation system the purge stream with the C₈+ product fraction to form a first intermediate stream; separating the first intermediate stream to provide a C₆₋₁₀ linear alpha olefin fraction, and a heavy stream comprising a C₁₂+ product fraction and the polymer byproduct; and recycling at least a portion of the C₆₋₁₀ linear alpha olefin fraction to a reactor flushing system.

The above described and other features are exemplified by the following FIGURE and detailed description.

### BRIEF DESCRIPTION OF THE DRAWING

The following FIGURE is an exemplary embodiment wherein like elements are numbered alike.

FIG. 1 shows a schematic illustration of a method for flushing an oligomerization reactor with flushing medium comprising C₈₋₁₀ linear alpha olefins.

### DETAILED DESCRIPTION

Described herein is a process for flushing an oligomerization reactor. It was unexpectedly discovered that employing a flushing medium comprising C₆ linear alpha olefins, C₈ linear alpha olefins, C₁₀ linear alpha olefins, or a combination comprising at least one of the foregoing can effectively clean the oligomerization reactor. In particular, the flushing medium used according to the process of the present disclosure can advantageously remove various polymer byproducts formed during an oligomerization reaction. Furthermore, the flushing medium can be separated from the polymer byproducts, heavy linear alpha olefin fractions (e.g., C₁₂+ linear alpha olefins), and the like, and at least a portion of the flushing medium can be recycled back to the reactor flushing system and used for subsequent cleanings of the oligomerization reactor, which represents the build-up of an open flushing cycle over the oligomerization reactor system. Reprocessing or disposal of the flushing medium can be easily achieved within the oligomerization plant in that contaminated flushing medium is processed in the various sections following the reactor (e.g., in a separation system). As such, disposal is of the flushing medium is cost-effective, since no specific additional equipment for the reprocessing of the flushing medium is required.

Accordingly, one aspect of the present disclosure is a process for flushing an oligomerization reactor. The process comprises flushing the oligomerization reactor with a flushing medium comprising C₆ linear alpha olefins, C₈ linear alpha olefins, C₁₀ linear alpha olefins, or a combination comprising at least one of the foregoing, to provide a purge stream from the flushing cycle comprising the C₆ linear alpha olefins, C₈ linear alpha olefins, C₁₀ linear alpha olefins, or a combination comprising at least one of the foregoing, and a polymer byproduct of an oligomerization reaction.

In some embodiments, the flushing medium comprises C₆ linear alpha olefins. In some embodiments, the flushing medium comprises C₈ linear alpha olefins. In some embodiments, the flushing medium comprises C₁₀ linear alpha olefins. In some embodiments, the flushing medium preferably comprises C₈₋₁₀ linear alpha olefins. In some embodiments, the flushing medium consists of C₆ linear alpha olefins, C₈ linear alpha olefins, C₁₀ linear alpha olefins, or a combination comprising at least one of the foregoing.

In some embodiments, the flushing medium excludes a solvent. For example, the flushing medium can include less than 1 weight percent, or less than 0.5 weight percent, or less than 0.1 weight percent of a solvent. Preferably, no solvent is present in the flushing medium. In particular, the flushing medium can exclude a solvent that is used in the oligomerization reactor, for example, during an oligomerization reaction. For example, the flushing medium can exclude an aromatic solvent, for example, a solvent selected from toluene, xylenes, benzene, or a combination comprising at least one of the foregoing. In some embodiments, the flushing medium excludes C₁₂+ linear alpha olefins, for example, the flushing medium can exclude C₁₂-C₁₈ linear alpha olefins. For example, the flushing medium can include less than 1 weight percent, or less than 0.5 weight percent, or less than 0.1 weight percent of C₁₂+ linear alpha olefins. Preferably, no C₁₂+ linear alpha olefins are present in the flushing medium.

The polymer byproduct can include polyethylene. Polyethylene as used herein refers to relatively high molecular weight polymers of ethylene, and includes copolymers. The term polyethylene can also include linear, branched, and crosslinked polyethylenes. In some embodiments, the polyethylene byproduct can have a weight average molecular weight (Mw) of 50,000 to 10,000,000 Daltons. In some embodiments, the purge stream from the flushing cycle can further comprise heavy fractions (e.g., greater than C₁₂ olefins), waxes, and the like, or a combination comprising at least one of the foregoing, in addition to the polyethylene.

Flushing the oligomerization reactor can be under any desirable conditions. For example, in some embodiments, flushing the oligomerization reactor can be at a temperature of 150 to 200° C., for example 170 to 200° C. In some embodiments, flushing the oligomerization reactor can be at a temperature of 170° C. In some embodiments, flushing the oligomerization reactor can be at a pressure high enough to avoid vaporization of the flushing medium during the flushing procedure. The flow rate of the flushing medium can be such that ensures turbulent flow in the reactor. The purge stream from the open flushing cycle can be high enough to maintain the concentration of polymers in the flushing medium below its limit of solubility to avoid precipitation of polymers. A desirable make-up stream will be routed to the flushing system to maintain the required system volume.

Once introduced to the reactor, the flushing medium is distributed throughout the reactor and its equipment to dissolve fouled material present as solid deposits. As mentioned above, the fouled material can include a polymer byproduct, for example polyethylene, present on the reactor walls. As the polymer byproduct is soluble in the flushing medium, the contaminated flushing medium can be discharged from the reactor, effectively cleaning the oligomerization reactor.

The process for flushing the oligomerization reactor can optionally further comprise contacting, in a separation system, the purge stream from the flushing cycle with a C₈+ product fraction from an olefin oligomerization process to form a first intermediate stream, separating the first intermediate stream to provide a C₆₋₁₀ linear alpha olefin fraction, preferably a C₈₋₁₀ linear alpha olefin fraction, and a heavy stream comprising a C₁₂+ product fraction and the polymer byproduct, and recycling at least a portion of the C₆₋₁₀ linear alpha olefin fraction, preferably the C₈₋₁₀ linear alpha olefin fraction, to a reactor flushing system. In some embodiments, the separation system can comprise one or more distillation columns, a thin film vaporizer, a wiped film vaporizer, a falling film vaporizer, or a combination comprising at least one of the foregoing.

In some embodiments, the process can alternatively comprise combining the purge stream from the flushing cycle directly with a C₁₂+ product fraction from an oligomerization process. In some embodiments, the process can further comprise recovering the C₆ linear alpha olefins, C₈ linear alpha olefins, Cm linear alpha olefins, or a combination comprising at least one of the foregoing from the purge stream. Recovering the C₆₋₁₀ linear alpha olefins from the purge stream can be by, for example, one or more distillation columns, a thin film vaporizer, a wiped film vaporizer, a falling film vaporizer, or a combination comprising at least one of the foregoing.

In some embodiments, the process can optionally comprise directly disposing of the purge stream to plant battery limits. Disposing of the purge stream can be by, for example, incineration.

In some embodiments, flushing the oligomerization reactor can occur after the reactor has been used to perform an oligomerization of an olefin to provide a linear alpha olefin product. In some embodiments, the olefin is ethylene. The linear alpha olefin products can generally be addition products containing greater than or equal to two ethylene units, but not as many ethylene units as in the relatively high molecular weight addition product referred to as polyethylene. In some embodiments, the oligomerization is a selective oligomerization process, for example a selective ethylene trimerization or tetramerization process. In some embodiments, the oligomerization is a trimerization process. In some embodiments, the linear alpha olefin products comprise C₄₋₁₂ linear alpha olefins. In some embodiments, the linear alpha olefin products comprise C₄₋₈ linear alpha olefins. For example, the linear alpha olefins can include at least one of 1-butene, 1-hexene, or 1-octene.

In an embodiment, the process for flushing an oligomerization reactor can comprise flushing the oligomerization reactor with a flushing medium comprising C₆ linear alpha olefins, C₈ linear alpha olefins, C₁₀ linear alpha olefins, or a combination comprising at least one of the foregoing, to provide a purge stream comprising the C₆ linear alpha olefins, C₈ linear alpha olefins, C₁₀ linear alpha olefins, or a combination comprising at least one of the foregoing, and a polymer byproduct. In a separation system, the purge stream contacts a C₈+ product fraction from an olefin oligomerization process to form a first intermediate stream. The process further comprises separating the first intermediate stream to provide a C₆₋₁₀ linear alpha olefin fraction, preferably a C₈₋₁₀ linear alpha olefin fraction, and a heavy stream comprising a C₁₂+ product fraction and the polymer byproduct, and recycling at least a portion of the C₆₋₁₀ linear alpha olefin fraction, preferably the C₈₋₁₀ linear alpha olefin fraction, to a reactor flushing system.

The process for flushing an oligomerization reactor can advantageously be used in conjunction with any known olefin oligomerization process. Accordingly, another embodiment is a process for the oligomerization of an olefin. The process can comprise feeding an olefin, a solvent, and a catalyst composition into a reactor and oligomerizing the olefin in the reactor to form a reaction product stream comprising linear alpha olefins.

The olefin can include any compound having 2 to 30 carbon atoms and at least one olefinic double bond. For example, the olefin can be ethylene, propylene, 1-butene, 2-butene, isobutylene, 1-pentene, 2-pentene, 1-hexene, 2-hexene, 3-hexene, 1-heptene, 2-heptene, 3-heptene, and the like, or a combination comprising at least one of the foregoing. In some embodiments, the olefin is ethylene.

The solvent can be any organic solvent capable of dissolving the reaction components. The solvent can be non-reactive with the catalyst composition. Examples of desirable organic solvents can include, but are not limited to, aromatic hydrocarbon solvents which can be unsubstituted or substituted, for example, toluene, benzene, ethyl benzene, xylene, mesitylene, monochlorobenzene, dichlorobenzene, chlorotoluene, aliphatic paraffin hydrocarbons, for example, pentane, hexane, heptane, octane, nonane, decane, alicyclic hydrocarbon compounds, for example, cyclohexane, decahydronaphthalene, and halogenated alkanes, for example, dichloroethane and dichlorobutane, or a combination comprising at least one of the foregoing. In some embodiments, the solvent can be toluene, xylene, mesitylene, ethyl benzene, n-pentane, n-hexane, cyclohexane, or a combination comprising at least one of the foregoing.

The catalyst composition can be any catalyst system that can oligomerize ethylene. For example, the catalyst composition can include a chromium source, a heteroatomic multidentate ligand, and an activator, also known as a co-catalyst. A catalyst modifier is not required, but is also preferably present.

The chromium compound can be an organic or inorganic salt, coordination complex, or organometallic complex of Cr(II) or Cr(III). In some embodiments the chromium compound is CrCl₃(tetrahydrofuran)₃, Cr(III)acetylacetonate, Cr(III)octanoate, chromium hexacarbonyl, Cr(III)-2-ethylhexanoate, benzene(tricarbonyl)-chromium, or Cr(III)chloride. A combination of different chromium compounds can be used.

The heteroatomic multidentate ligand includes two or more heteroatoms (P, N, O, S, As, Sb, Bi, O, S, or Se) that can be the same or different, wherein the two or more heteroatoms are linked via a linking group. The linking group is a C₁₋₆ hydrocarbylene group or one of the foregoing heteroatoms. Any of the heteroatoms in the ligand can be substituted to satisfy the valence thereof, with a hydrogen, halogen, C₁₋₁₈ hydrocarbyl group, C₁₋₁₀ hydrocarbylene group linked to the same or different heteroatoms to form a heterocyclic structure, amino group of the formula NR^{a}R^{b} wherein each of R^{a} and R^{b} is independently hydrogen or a C₁₋₁₈ hydrocarbyl group, a silyl group of the formula SiR^{a}R^{b}R^{c} wherein each of R^{a}, R^{b}, and R^{c} is independently hydrogen or a C₁₋₁₈ hydrocarbyl group, or a combination comprising at least one of the foregoing sub stituents. The heteroatoms of the multidentate ligand are preferably a combination comprising phosphorus with nitrogen and sulfur or a combination comprising phosphorous and nitrogen, linked by at least one additional phosphorus or nitrogen heteroatom. In certain embodiments, the ligand can have the backbone PNP, PNPN, NPN, NPNP, NPNPN, PNNP, or cyclic derivatives containing these backbones wherein one or more of the heteroatoms is linked by a C₁₋₁₀ hydrocarbylene to provide a heterocyclic group. A combination of different ligands can be used.

In some embodiments, the ligand has the backbone PNPNH, which as used herein has the general structure R¹R²P—N(R³)—P(R⁴)—N(R⁵)—H wherein each of R′, R², R³, R⁴, and R⁵ is independently a hydrogen, halogen, C₁₋₁₈ hydrocarbyl group, amino group of the formula NR^{a}R^{b} wherein each of R^{a} and R^{b} is independently hydrogen or a C₁₋₁₈ hydrocarbyl group, a silyl group of the formula SiR^{a}R^{b}R^{c} wherein each of R^{a}, R^{b}, and R^{c} is independently hydrogen or a C₁₋₁₈ hydrocarbyl group, or two of R′, R², R³, R⁴, R⁵, R^{a}, or R^{b} taken together are a substituted or unsubstituted C₁₋₁₀ hydrocarbylene group linked to the same or different heteroatoms to form a heterocyclic structure. Exemplary ligands having a heterocyclic structure include the following

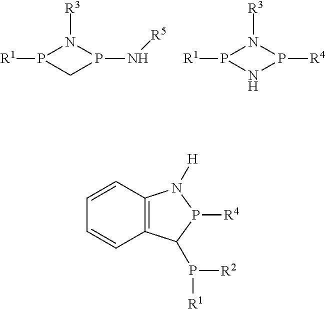

wherein R¹, R², R³, R⁴, R⁵ are as described above. In a specific embodiment, each R¹, R², R³, R⁴, R⁵ are independently hydrogen, substituted or unsubstituted C₁-C₈ alkyl, substituted or unsubstituted C₆-C₂₀ aryl, more preferably unsubstituted C₁-C₆ alkyl or unsubstituted C₆-C₁₀ aryl. A specific example of the ligand is (phenyl)₂PN(iso-propyl)P(phenyl)N(iso-propyl)H, commonly abbreviated Ph₂PN(i-Pr)P(Ph)NH(i-Pr).

Activators can include aluminum compounds, for example a tri(C₁-C₆alkyl) aluminum such as triethyl aluminum, (C₁-C₆ alkyl) aluminum sesquichloride, di(C₁-C₆alkyl) aluminum chloride, or (C₁-C₆-alkyl) aluminum dichloride, or an aluminoxane such as methylaluminoxane (MAO). Each alkyl group can be the same or different, and in some embodiments is methyl, ethyl, isopropyl, or isobutyl. A combination of different activators can be used.

As is known in the art, the modifier can modify the activator, and serve as a chlorine source. Modifiers can include an ammonium or phosphonium salt of the type (H₄E)X, (H₃ER)X, (H₂ER₂)X, (HER₃)X, or (ER₄)X wherein E is N or P, X is Cl, Br, or I, and each R is independently a C₁-C₂₂ hydrocarbyl, preferably a substituted or unsubstituted C₁-C₁₆-alkyl, C₂-C₁₆-acyl, or substituted or unsubstituted C₆-C₂O-aryl. In some embodiments the modifier is dodecyltrimethylammonium chloride or tetraphenylphosphonium chloride.

The catalyst composition is often pre-formed (i.e., formed prior to contacting other reaction components in the oligomerization reactor), for example by combining the components in a solvent before contacting with ethylene in an oligomerization process. Examples of solvents that can be used include toluene, benzene, ethylbenzene, cumenene, xylenes, mesitylene, C₄-C₁₅ paraffins, cyclohexane, C₄-C₁₂ olefins such as butene, hexene, heptene, octene, or ethers or multiethers such as diethylether, tetrahydrofuran, dioxane, di(C₁-C₈ alkyl)ethers. In some embodiments the solvent is an aromatic solvent such as toluene.

The type of each component selected for use in the catalyst composition and relative amount of each component depend on the desired product and desired selectivity. In some embodiments, the concentration of the chromium compound is 0.01 to 100 millimole per liter (mmol/1), or 0.01 to 10 mmol/1, or 0.01 to 1 mmol/1, or 0.1 to 1.0 mmol/1; and the mole ratio of multidentate ligand:Cr compound:activator is 0.1:1:1 to 10:1:1,000, or 0.5:1:50 to 2:1:500, or 1:1:100 to 5:1:300. Suitable catalyst systems are described, for example, in EP2489431 Bl; EP2106854 Bl; and WO2004/056479.

The above described components can be fed into a reactor. The reactor can be any oligomerization reactor. For example, the reactor can be a loop reactor, a plug-flow reactor, a bubble column reactor, or a tubular reactor.

The process can further comprise oligomerizing the olefin in the reactor to form a reaction product stream. The reaction product stream can comprise linear alpha olefins, the solvent, and the catalyst composition. The linear alpha olefins made by the process disclosed herein can generally be addition products containing greater than or equal to two ethylene units, but not as many ethylene units as in the relatively high molecular weight addition product referred to as polyethylene. In some embodiments, the process can be adapted to be a selective oligomerization process, for example a selective ethylene trimerization or tetramerization process. In some embodiments, the linear alpha olefins comprise C₄₋₁₂ linear alpha olefins. In some embodiments, the linear alpha olefins comprise C₄₋₈ linear alpha olefins. For example, the linear alpha olefins can include at least one of 1-butene, 1-hexene, or 1-octene.

Oligomerization can occur at temperatures of 10 to 200° C., for example, 20 to 100° C., for example, 50 to 90° C., for example, 55 to 80° C., for example, 60 to 70° C. Operating pressures can be 1 to 5 MegaPascals (MPa), for example, 2 to 4 MPa. The process can be continuous and mean residence times can be 10 minutes to 20 hours, for example 30 minutes to 4 hours, for example, 1 to 2 hours. Residence times can be chosen so as to achieve the desired conversion at high selectivity.

Subsequent to oligomerizing the olefin in the reactor, the reactor can be flushed with a flushing medium comprising C₆ linear alpha olefins, C₈ linear alpha olefins, C₁₀ linear alpha olefins, or a combination comprising at least one of the foregoing, to provide a purge stream. The purge stream from the flushing cycle can comprise the C₆ linear alpha olefins, C₈ linear alpha olefins, C₁₀ linear alpha olefins, or a combination comprising at least one of the foregoing, and a polymer byproduct. In some embodiments, the polymer byproduct can comprise polyethylene. The purge stream is contacted with the C₈+ product fraction in a separation system, forming a first intermediate stream. The separation system can comprise one or more distillation columns, a thin film vaporizer, a wiped film vaporizer, a falling film vaporizer, or a combination comprising at least one of the foregoing. The first intermediate stream can be separated to provide a C₆₋₁₀ linear alpha olefin fraction, and a heavy stream comprising a C₁₂+ product fraction and the polymer byproduct. The process can further comprise recycling at least a portion of the C₆₋₁₀ linear alpha olefin fraction to a reactor flushing system. The reactor flushing system can be connected to the oligomerization reactor and can provide flushing medium to the reactor and can receive “contaminated” flushing medium (i.e., the purge stream) from the reactor.

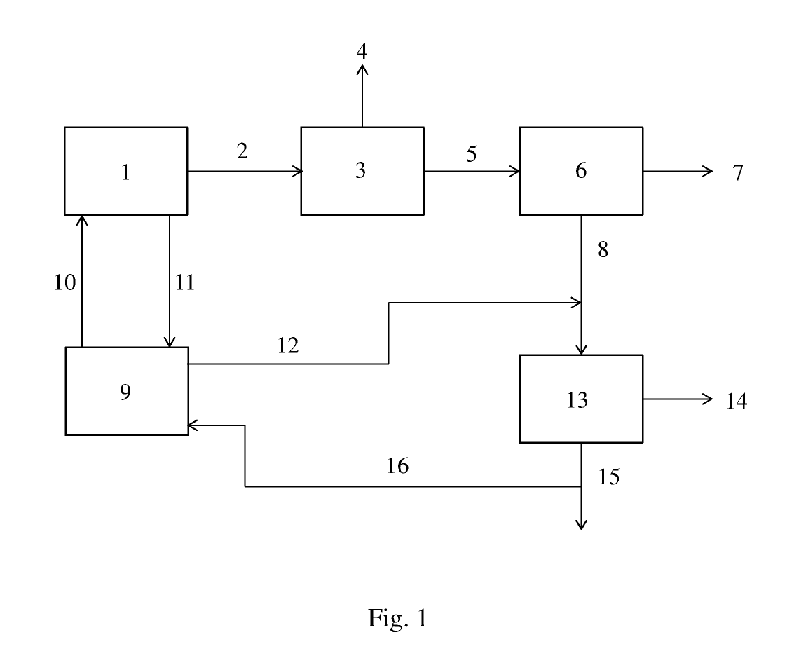

In an embodiment, the oligomerization and oligomerization reactor flushing processes can be carried out according to the process depicted in FIG. 1. An oligomerization reactor (1) can generate a reaction product stream (2) comprising the linear alpha olefins. The reaction product stream (2) can be subjected to a series of separation steps. In a first product separation step (3), C₄ linear alpha olefins can be separated and removed (4). The remaining reaction product stream (C₆+ linear alpha olefins) (5) can be transferred to a second product separation step (6), where C₆ linear alpha olefins can be separated and removed (7). The remaining C₈+ product fraction (8) can be removed from the second separator (6). The reactor (1) is connected to flushing system (9) which, subsequent to an oligomerization reaction, can provide the flushing medium (10) to the reactor (1). The flushing medium (10) can be distributed throughout the reactor (1) to dissolve fouled polymer byproducts of the oligomerization reaction, and the contaminated flushing medium (11) can be returned to the flushing system (9). The contaminated flushing medium can be transferred from the flushing system (9) as a purge stream (12), which can be combined with the C₈+ product fraction (8). The combined stream can be processed in C₈+ processing unit (13). A C₁₂+ fraction including the dissolved polymer byproducts can be separated as heavy stream (14), and removed for further processing or disposal. A C₈₋₁₀ product stream (15) can be recovered, and a portion returned (16) to the flushing system (9) to be used in subsequent reactor cleaning processes.

The present disclosure provides an improved process for the flushing of an oligomerization reactor. The use of the particular reactor flushing medium described herein can provide several advantageous features including reducing or eliminating the need for importing to the plant excess solvent typically required to clean a reactor, the installation of a separate additional process section for reprocessing of the flushing medium, and providing a cost-effective process for flushing an oligomerization reactor where at least a portion of the flushing medium can be recovered and recycled. Therefore, a substantial improvement in the flushing of an oligomerization reactor is provided.

The processes disclosed herein can be further illustrated by the following non-limiting examples.

### EXAMPLES

An ethylene trimerization plant for the production of 100,000 tons per year of 1-hexene is equipped with trimerization reactors which have to be flushed periodically. Flushing shall be performed with a C₈₋₁₀ fraction which is produced within the plant. The flowrate of the C₈₋₁₀ linear alpha olefin product fraction is 75 tons per hour (t/h), and the product fraction is heated in increments of about 15 Kelvin (K) by each cycle in a heat exchanger to a final temperature of 170° C. at a pressure of 8.5 bar(a). The product fraction is routed to the corresponding trimerization reactor. Within the flushing system, by means of a flushing cycle pump, an open C₈₋₁₀ cycle of 75 t/h over the trimerization reactor is established. Ethylene polymers which are deposited in the trimerization reactor are dissolved in the flushing medium. In order to limit the concentration of dissolved polymers in the flushing medium at a value below its limit of solubility, a continuous purge stream of 1.0 t/h is withdrawn from the flushing cycle. This purge stream is routed to the separation section of the 1-hexene plant, namely the C₁₀/C₁₂ separation unit. In this separation step, the purge stream is processed together with the C₈+ from the trimerization reactor outlet stream. The overhead product of the C₁₀/C₁₂ separation represents the C₈₋₁₀ by-product of the 1-hexene plant. A flow rate of 1.0 t/h of C₈₋₁₀ is used as a make-up stream in the flushing system, and the remainder is routed as C₈₋₁₀ by-product to plant battery limit. The bottoms product of the C₁₀/C₁₂ separation, comprising the C₁₂+ components produced in the trimerization reactor as well as the dissolved ethylene polymers recovered from the flushing system is routed as by-product to plant battery limit.

The processes disclosed herein include at least the following embodiments:

### Embodiment 1

A process for flushing an oligomerization reactor, comprising: flushing the oligomerization reactor with a flushing medium comprising C₆ linear alpha olefins, C₈ linear alpha olefins, C₁₀ linear alpha olefins, or a combination comprising at least one of the foregoing, to provide a purge stream comprising the C₆ linear alpha olefins, C₈ linear alpha olefins, C₁₀ linear alpha olefins, or a combination comprising at least one of the foregoing, and a polymer byproduct of an oligomerization reaction.

### Embodiment 2

The process of embodiment 1, further comprising contacting, in a separation system, the purge stream with a C₈+ product fraction from an olefin oligomerization process to form a first intermediate stream; separating the first intermediate stream to provide a C₆₋₁₀ linear alpha olefin fraction, and a heavy stream comprising a C₁₂+ product fraction and the polymer byproduct; and recycling at least a portion of the C₆₋₁₀ linear alpha olefin fraction to a reactor flushing system.

### Embodiment 3

The process of embodiment 1, further comprising combining the purge stream with a C₁₂+ product fraction from an oligomerization process.

### Embodiment 4

The process of embodiment 1 or embodiment 3, further comprising recovering the C₆ linear alpha olefins, C₈ linear alpha olefins, C₁₀ linear alpha olefins, or a combination comprising at least one of the foregoing from the purge stream.

### Embodiment 5

The process of any of embodiments 1, 3, or 4, further comprising disposing of the purge stream, preferably by incineration.

### Embodiment 6

The process of any of embodiments 1 to 5, wherein the flushing medium comprises C₆ linear alpha olefins.

### Embodiment 7

The process of any of embodiments 1 to 5, wherein the flushing medium comprises C₈ linear alpha olefins.

### Embodiment 8

The process of any of embodiments 1 to 5, wherein the flushing medium comprises C₁₀ linear alpha olefins.

### Embodiment 9

The process of any of embodiments 1 to 5, wherein the flushing medium comprises C₈₋₁₀ linear alpha olefins.

### Embodiment 10

The process of any of embodiments 1 to 9, wherein flushing the oligomerization reactor occurs after the reactor is used to perform an oligomerization of an olefin to provide a linear alpha olefin product.

### Embodiment 11

The process of embodiment 10, wherein the olefin is ethylene.

### Embodiment 12

The process of embodiment 10 or embodiment 11, wherein the oligomerization is a selective trimerization or tetramerization, preferably a selective trimerization.

### Embodiment 13

The process of any of embodiments 1 to 12, wherein the polymer byproduct comprises polyethylene.

### Embodiment 14

The process of any of embodiments 1 to 13, wherein the separation system comprises one or more distillation columns, a thin film vaporizer, a wiped film vaporizer, a falling film vaporizer, or a combination comprising at least one of the foregoing.

### Embodiment 15

The process of any of embodiments 1 to 14, wherein the flushing medium excludes a solvent, preferably wherein the flushing medium excludes a solvent that is used in the oligomerization reactor.

### Embodiment 16

The process of any of embodiments 1 to 15, wherein the flushing medium excludes a solvent selected from toluene, xylenes, benzene, and a combination comprising at least one of the foregoing.

### Embodiment 17

The process of any of embodiments 1 to 16, wherein the flushing medium excludes C₁₂+ linear alpha olefins, preferably wherein the flushing medium excludes C₁₂-C₁₈ linear alpha olefins.

### Embodiment 18

The process of any of claims 1 to 17, wherein flushing the oligomerization reactor is at a temperature of 150 to 200° C., preferably at a temperature of 170 to 200° C.

### Embodiment 19

A process for flushing an oligomerization reactor, comprising: flushing the oligomerization reactor with a flushing medium comprising C₆ linear alpha olefins, C₈ linear alpha olefins, C₁₀ linear alpha olefins, or a combination comprising at least one of the foregoing, to provide a purge stream comprising the C₆ linear alpha olefins, C₈ linear alpha olefins, C₁₀ linear alpha olefins, or a combination comprising at least one of the foregoing, and a polymer byproduct; contacting, in a separation system, the purge stream with a C₈+ product fraction from an olefin oligomerization process to form a first intermediate stream; separating the first intermediate stream to provide a C₆₋₁₀ linear alpha olefin fraction, and a heavy stream comprising a C₁₂+ product fraction and the polymer byproduct; and recycling at least a portion of the C₆₋₁₀ linear alpha olefin fraction to a reactor flushing system.

### Embodiment 20

A process for oligomerization of an olefin, comprising: oligomerizing the olefin in the reactor to form a reaction product stream comprising linear alpha olefins; separating a C₄₋₆ linear alpha olefin product fraction from the reaction product stream to provide a first stream comprising a C₈+ product fraction; subsequent to oligomerizing the olefin in the reactor, flushing the reactor with a flushing medium comprising C₆ linear alpha olefins, C₈ linear alpha olefins, Cm linear alpha olefins, or a combination comprising at least one of the foregoing, to provide a purge stream comprising the C₆ linear alpha olefins, C₈ linear alpha olefins, C₁₀ linear alpha olefins, or a combination comprising at least one of the foregoing, and a polymer byproduct; contacting in a separation system the purge stream with the C₈+ product fraction to form a first intermediate stream; separating the first intermediate stream to provide a C₆₋₁₀ linear alpha olefin fraction, and a heavy stream comprising a C₁₂+ product fraction and the polymer byproduct; and recycling at least a portion of the C₆₋₁₀ linear alpha olefin fraction to a reactor flushing system.

In general, the processes can alternatively comprise, consist of, or consist essentially of, any appropriate components herein disclosed. The processes can additionally, or alternatively, be formulated so as to be devoid, or substantially free, of any components, materials, ingredients, adjuvants, species or process steps used in the prior art compositions or processes or that are otherwise not necessary to the achievement of the function and/or objectives of the present invention. The endpoints of all ranges directed to the same component or property are inclusive and independently combinable. Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term. Reference throughout the specification to “one embodiment”, “another embodiment”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs.

As used herein, the term “hydrocarbyl” includes groups containing carbon, hydrogen, and optionally one or more heteroatoms (e.g., 1, 2, 3, or 4 atoms such as halogen, 0, N, S, P, or Si). “Alkyl” means a branched or straight chain, saturated, monovalent hydrocarbon group, e.g., methyl, ethyl, i-propyl, and n-butyl. “Aryl” means a monovalent, monocyclic, or polycyclic aromatic group (e.g., phenyl or naphthyl). “Substituted” means that the compound or group is substituted with at least one (e.g., 1, 2, 3, or 4) substituents instead of hydrogen, where each substituent is independently nitro (—NO₂), cyano (—CN), hydroxy (—OH), halogen, thiol (—SH), thiocyano (—SCN), C₁₋₆ alkyl, C₂-6 alkenyl, C₂-6 alkynyl, C₁₋₆ haloalkyl, C₁₋₉ alkoxy, C₁₋₆ haloalkoxy, C₃₋₁₂ cycloalkyl, C₅₋₁₈ cycloalkenyl, C₆₋₁₂ aryl, C₇₋₁₃ arylalkylene (e.g., benzyl), C₇₋₁₂ alkylarylene (e.g., toluyl), C₄₋₁₂ heterocycloalkyl, C₃₋₁₂ heteroaryl, C₁₋₆ alkyl sulfonyl (—S(═O)₂-alkyl), C₆₋₁₂ arylsulfonyl (—S(═O)₂-aryl), or tosyl (CH₃C₆H₄SO₂—), provided that the substituted atom's normal valence is not exceeded, and that the substitution does not significantly adversely affect the manufacture, stability, or desired property of the compound. When a compound is substituted, the indicated number of carbon atoms is the total number of carbon atoms in the group, including those of the substituent(s).

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A process for flushing an oligomerization reactor, comprising:
flushing the oligomerization reactor with a flushing medium comprising C₆ linear alpha olefins, C₈ linear alpha olefins, C₁₀ linear alpha olefins, or a combination comprising at least one of the foregoing, to provide a purge stream comprising the C₆ linear alpha olefins, C₈ linear alpha olefins, C₁₀ linear alpha olefins, or a combination comprising at least one of the foregoing, and a polymer byproduct of an oligomerization reaction.

2. The process of claim 1, further comprising
contacting, in a separation system, the purge stream with a C₈+ product fraction from an olefin oligomerization process to form a first intermediate stream;
separating the first intermediate stream to provide a C₆₋₁₀ linear alpha olefin fraction, and a heavy stream comprising a C₁₂+ product fraction and the polymer byproduct; and
recycling at least a portion of the C₆₋₁₀ linear alpha olefin fraction to a reactor flushing system.

3. The process of claim 1, further comprising combining the purge stream with a C₁₂+ product fraction from an oligomerization process.

4. The process of claim 1, further comprising recovering the C₆ linear alpha olefins, C₈ linear alpha olefins, C₁₀ linear alpha olefins, or a combination comprising at least one of the foregoing from the purge stream.

5. The process of claim 1, further comprising disposing of the purge stream, preferably by incineration.

6. The process of claim 1, wherein the flushing medium comprises C₆ linear alpha olefins.

7. The process of claim 1, wherein the flushing medium comprises C₈ linear alpha olefins.

8. The process of claim 1, wherein the flushing medium comprises C₁₀ linear alpha olefins.

9. The process of claim 1, wherein the flushing medium comprises C₈₋₁₀ linear alpha olefins.

10. The process of claim 1, wherein flushing the oligomerization reactor occurs after the reactor is used to perform an oligomerization of an olefin to provide a linear alpha olefin product.

11. The process of claim 10, wherein the olefin is ethylene.

12. The process of claim 10, wherein the oligomerization is a selective trimerization or tetramerization, preferably a selective trimerization.

13. The process of claim 1, wherein the polymer byproduct comprises polyethylene.

14. The process of claim 1, wherein the separation system comprises one or more distillation columns, a thin film vaporizer, a wiped film vaporizer, a falling film vaporizer, or a combination comprising at least one of the foregoing.

15. The process of claim 1, wherein the flushing medium excludes a solvent.

16. The process of claim 1, wherein the flushing medium excludes a solvent selected from toluene, xylenes, benzene, and a combination comprising at least one of the foregoing.

17. The process of claim 1, wherein the flushing medium excludes C₁₂+ linear alpha olefins.

18. The process of claim 1, wherein flushing the oligomerization reactor is at a temperature of 150 to 200° C.

19. A process for flushing an oligomerization reactor, comprising:
flushing the oligomerization reactor with a flushing medium comprising C₆ linear alpha olefins, C₈ linear alpha olefins, C₁₀ linear alpha olefins, or a combination comprising at least one of the foregoing, to provide a purge stream comprising the C₆ linear alpha olefins, C₈ linear alpha olefins, C₁₀ linear alpha olefins, or a combination comprising at least one of the foregoing, and a polymer byproduct;
contacting, in a separation system, the purge stream with a C₈+ product fraction from an olefin oligomerization process to form a first intermediate stream;
separating the first intermediate stream to provide a C₆₋₁₀ linear alpha olefin fraction, and a heavy stream comprising a C₁₂+ product fraction and the polymer byproduct; and
recycling at least a portion of the C₆₋₁₀ linear alpha olefin fraction to a reactor flushing system.

20. A process for oligomerization of an olefin, comprising:
oligomerizing the olefin in the reactor to form a reaction product stream comprising linear alpha olefins;
separating a C₄₋₆ linear alpha olefin product fraction from the reaction product stream to provide a first stream comprising a C₈+ product fraction;
subsequent to oligomerizing the olefin in the reactor, flushing the reactor with a flushing medium comprising C₆ linear alpha olefins, C₈ linear alpha olefins, C₁₀ linear alpha olefins, or a combination comprising at least one of the foregoing, to provide a purge stream comprising the C₆ linear alpha olefins, C₈ linear alpha olefins, C₁₀ linear alpha olefins, or a combination comprising at least one of the foregoing, and a polymer byproduct;
contacting in a separation system the purge stream with the C₈+ product fraction to form a first intermediate stream;
separating the first intermediate stream to provide a C₆₋₁₀ linear alpha olefin fraction, and a heavy stream comprising a C₁₂+ product fraction and the polymer byproduct; and
recycling at least a portion of the C₆₋₁₀ linear alpha olefin fraction to a reactor flushing system.
